(19) United States
(12) Patent Application Publication
(10) Pub. No.: US 2006/0173711 A1
(43) Pub. Date: Aug. 3, 2006

(54) Patient health status data management method and system

(75) Inventor: George M. Rapier III, San Antonio, TX (US)

Correspondence Address: Gregg R. Kronenberger;JENKENS & GILCHRIST, A PROFESSIONAL CORPORATION, Suite 3700, 1445 Ross Avenue, Dallas, TX, 75202, US

(73) Assignee: WELLMED MEDICAL MANAGEMENT, INC.

(21) Appl. No.: 11/223,192

(22) Filed: Sep. 9, 2005

Related U.S. Application Data
(60) Provisional application No. 60/608,228, filed on Sep. 9, 2004.

(51) Int. Cl. G06Q 10/00; A61B 5/00

(52) U.S. Cl. 705/2; 600/300

(57) ABSTRACT

The present invention comprises a method for managing patient health status data. More specifically, a method and system is disclosed for reviewing and analyzing patient health related information and ensuring that it contains all appropriate health status data for the patient. All of a patient's health related information is gathered from available sources. That information is then analyzed to determine whether the appropriate health status data is present in the patient's health information. All appropriate health status data that is unreported in or inconsistent with the patient's health related information is identified. The patient's physician is then presented with suggested supplemental appropriate health status data and the physician's approval or rejection of the suggestion is sought. After the physician's approval or rejection, the patient's health status data is supplemented accordingly, and the data is compiled for future use or processing.

### CROSS-REFERENCE TO RELATED APPLICATION

This non-provisional application claims priority based upon prior U.S. Provisional Patent Application Ser. No. 60/608,228, filed Sep. 9, 2004, in the name of George M. Rapier, III, entitled “Healthcare Payment Method and System,” the entirety of which is incorporated herein by reference.

### FIELD OF THE INVENTION

The disclosures herein relate generally to a patient health status data management method and system. More specifically, a method and system is disclosed for reviewing and analyzing patient health related information and ensuring that they contain all appropriate health status data for the patient.

### BACKGROUND AND SUMMARY OF THE INVENTION

Health care providers (sometimes referred to simply as “providers”) generally provide health care services, and receive payment for those services, under one of two models: fee for service; or managed care. In either model, the entities that pay for the providers' services (“payors”) generally require as a condition of payment that the providers use established coding systems to identify health related information for each patient. This information can include, for example, the services provided the patient, the patient's health status, and diagnoses of the patient's condition.

Under the fee for service model, the provider will generally charge and be paid for the type of services rendered or performed. Thus, the charge, and therefore the payment, will vary from patient to patient based on the care provided to the patient. The costs of these services are determined by market forces. In simplest terms, in the fee for service environment, the provider is paid based on what was done for the patient.

The managed care model has traditionally paid providers on a fixed per capita (or “capitated”) basis per patient. Historically, any adjustments to such capitated payments were based on patient demographics like age and sex and other factors. In the same simple terms used above in describing the fee for service model, the managed care provider is paid based on who the patients are.

Medicare/Medicaid is the federal system for providing health care to elderly, indigent, and disabled persons. The Medicare/Medicaid system is administered by the Center for Medicare/Medicaid Services (“CMS”).

Under the Medicare/Medicaid system, health care is provided to patients in both the fee for service and the managed care format. Medicare/Medicaid managed care is provided through providers who are under contracts with health maintenance organizations (“HMOs”) which obtain payment from CMS.

CMS has for many years required providers, as a condition of payment, to use coding systems to describe the health care services rendered. If a provider does not use the correct codes in its submission, the provider will not be paid appropriately for the services provided, and may not be paid at all. Many private health care payors also require providers to use codes to identify the services for which they seek payment.

Prior to 2004, the data submission required by CMS was identical for both fee for service and managed care providers. CMS generally required providers to submit codes describing the procedures performed on the patient and the appropriate corresponding codes describing the patient's diagnoses.

One such diagnosis coding system the International Classification of Diseases. The Edition currently in use in the United States is the 9th Edition (“ICD9”). At present, there are approximately 15,000 ICD9 diagnosis codes.

Prior to 2004, Medicare/Medicaid managed care capitation payments were based solely on the patient's demographics like age, sex, and other factors. This payment methodology did not take into account the illness, severity of illness, or other co-morbid conditions of the patient.

In response to the federal Balanced Budget Act of 1997, however, the CMS changed its payment methodology for managed care. The changes are being phased in over four years beginning January 2004. CMS's goal in changing the payment methodology is to pay providers in a manner and amount appropriate to the acuity of the patient.

The change to the CMS methodology is the creation of the Risk Adjusted Payment System (“RAPS”). In general terms, CMS now pays managed care providers based on the health status of the patient.

The new methodology is based on CMS's understanding, from accumulating years of patient data, that certain patient conditions pose different, and quantifiable, health risks. Thus, CMS identified approximately 4,000 ICD9 diagnosis codes that indicate conditions that can have a significant effect on health status (“RAPS codes” or “RAPS diagnoses”). The RAPS contemplates that if providers are fairly compensated for appropriately addressing the conditions identified by the RAPS diagnoses, then the health status of those patients will be better supervised, and overall health care costs will decline. A necessary corollary to this approach is that patients whose health status are not at risk will generally require less care, and thus managed care providers will be paid less for those patients. Stated simply, under RAPS, providers will be paid based on what is wrong with the patient. This is a very significant change in the management of patient care.

To implement RAPS, CMS classified the RAPS diagnoses into about 62 Hierarchical Condition Categories (“HCCs”), each of which is assigned a weighting factor that serves to adjust the capitation payment for the patient. Some of the weighting factors are additive, such that a patient with RAPS diagnoses in more than one HCC will justify an increase in payment equal to the sum of two HCC weighting factors. Thus, providers who appropriately diagnose patients with RAPS diagnoses and appropriately address these diagnoses will receive increased payments appropriate to the patient's health status.

Under the RAPS payment methodology, providers must do more than simply identify the appropriate ICD9 code and HCC for the patient's condition in order to receive the RAPS adjustment to the payment for the patient. The provider must also: (a) in a face-to-face meeting with the patient, address all of the RAPS diagnoses and recommend a care plan; and (b) follow up with the patient on the care plan on at least an annual basis. In addition, since capitation rates are only changed annually, providers require a method or system not only to capture current health status data, but also to track that data from year to year.

Thirty percent of what CMS paid Medicare managed care providers for calendar year 2004 was based on RAPS. That proportion increased to 50% in 2005, and will further increase to 75% in 2006. Beginning in January 2007, all payments to these providers will be based on RAPS. Those providers who do not understand and successfully adapt to the changed methodology will at best lose competitive advantage; at worst, they will have difficulty remaining viable.

Currently, the RAPS payment methodology only applies to Medicare managed care services. It is expected, however, that the methodology will be adopted for Medicaid managed care, as well as the private health care industry. It has been the experience in the health care industry that the private sector often adopts federal procedures, as it is more efficient for both providers and payors to standardize payment processes.

It is also anticipated that the risk adjusted payment concept could be embraced by other entities, such as large corporations that self-insure their health plans. Applied correctly, the concept can be used to enhance an entity's ability to track patients' care and health status and control costs.

In addition, tracking the health status data in the manner necessary for the risk adjusted payment concept enables any entity using the concept to manipulate and analyze that data for useful purposes other than payment. For example, the data can be used for quality improvement, quality control, analysis of treatment protocols, disease tracking, and wellness management.

What is needed, therefore, is a system and method to review and monitor patient health related data, capture all information necessary to describe the patient's health status, maintain and update such information, and report or otherwise produce such information for appropriate purposes.

### BRIEF DESCRIPTION OF THE DRAWINGS

A better understanding of the system and method of the present invention may be had by reference to the drawings or figures, wherein:

FIG. 1 is a flowchart providing a general overview of the present invention;

FIG. 2 is an exemplary screen view showing detail of a patient information record in a software application for facilitating the analysis conducted in a preferred embodiment of the present invention;

FIG. 3 is an exemplary screen view showing detail of a means for suggesting a patient diagnosis in a software application for facilitating the analysis conducted in a preferred embodiment of the present invention;

FIG. 4 is an exemplary screen view showing detail of diagnosis choices that may be made available to a user for suggesting patient diagnosis in a software application for facilitating the analysis conducted in a preferred embodiment of the present invention;

FIG. 5 is an exemplary screen view showing detail of the choices that may be made available to a user for identifying the health related information that justifies a suggested diagnosis in a software application for facilitating the analysis conducted in a preferred embodiment of the present invention;

FIG. 6 is exemplary form suggesting patient health status data to a physician generated by a software application for facilitating the analysis conducted in a preferred embodiment of the present invention;

FIG. 7 is an exemplary screen view showing detail of tracking by patient whether a physician has accepted or rejected suggested health status data in a software application for facilitating the analysis conducted in a preferred embodiment of the present invention;

FIG. 8 is an exemplary report tracking by physician the status of physician response to the suggestion of health status data generated by a software application for facilitating the analysis conducted in a preferred embodiment of the present invention;

FIG. 9 is an exemplary report tracking by nurse the status of nurse response to the suggestion of health status data generated by a software application for facilitating the analysis conducted in a preferred embodiment of the present invention;

FIG. 10 is an exemplary screen view showing detail of various reports that may be generated by a software application for facilitating the analysis conducted in a preferred embodiment of the present invention; and

FIG. 11 is an exemplary report showing audit nurse productivity generated by a software application for facilitating the analysis conducted in a preferred embodiment of the present invention.

### DETAILED DESCRIPTION OF SPECIFIC EMBODIMENTS

While the invention is susceptible to various modifications and alternative forms, specific embodiments have been shown by way of example and will be described in detail herein. However, it should be understood that the invention is not intended to be limited to the particular forms disclosed. Rather, the invention is to cover all modifications, equivalents, and alternatives falling within the spirit and scope of the invention as defined by the appended claims.

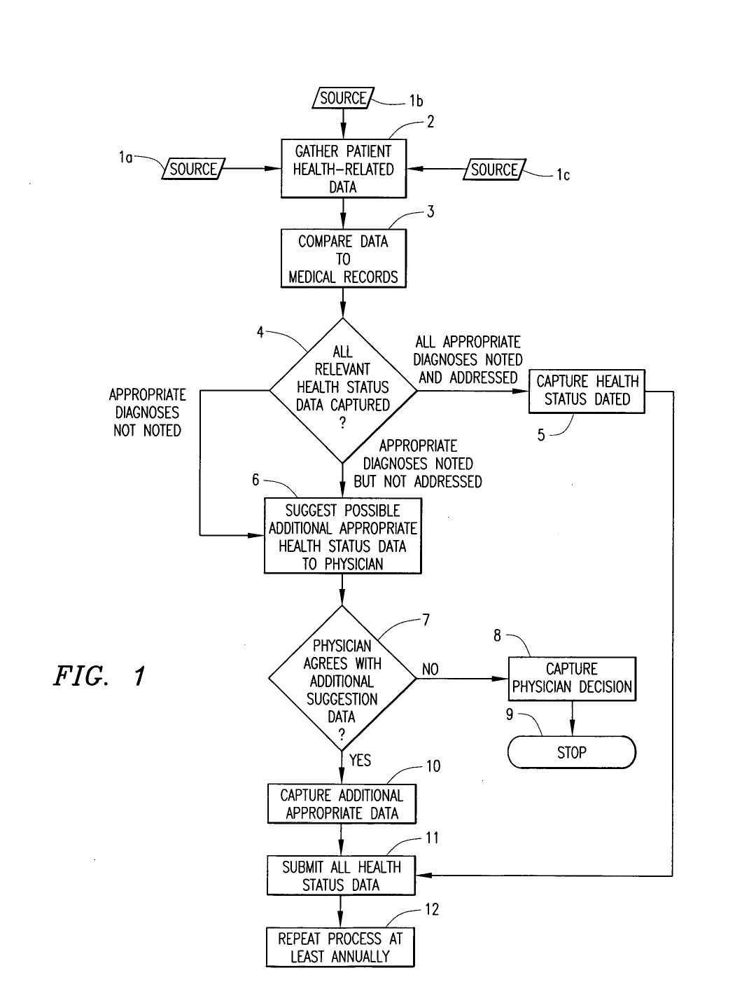

Referring to FIG. 1, patient health related information is obtained from all available sources 1a-1c, including health care providers, health plans, pharmacy benefit managers, physician billing records, and laboratory reports where available. Said information could include, for example, patient medical records, paid claims, encounter data, pharmacy benefit data, physician billing data, hospital paid claims, and member demographics. This information is then gathered 2 for each patient.

In one embodiment in which patient information is maintained in an electronic medical records system, hospital or health care provider information system software, or similar application, said patient information is gathered by evaluating it with matching software to correctly match all information to the correct patient using a master patient index. The matching software will match the vast majority of the information to the master patient index, but some manual evaluation is sometimes needed to review specific, difficult to match, sets of data. All the information is then sorted and transferred to a database application that will be used by nurse reviewers for analysis of the information, preparation of suggested supplemental data to the physician, preparation of follow up reports and letters, and submission of health status data to the health plan payor or the Center for Medicare/Medicaid Services (“CMS”).

The focus of the analysis 3 of the health related information is to identify all relevant patient health status data. In the current RAPS methodology, relevant patient health status data must include appropriate International Classification of Diseases, 9th Edition (“ICD9”) Risk Adjusted Payment System (“RAPS”) diagnoses and evidence that the physician has appropriately addressed all diagnoses.

In a preferred embodiment, a nurse reviewer with significant clinical experience and training in coding systems analyzes each patient's health related information. In another embodiment, this analysis may be performed at least in part by a software application. For ease of discussion, reference is made to a nurse reviewer conducting the analysis throughout the majority of this disclosure.

In one embodiment, the analysis can be performed at the office or facility of the custodian of the patient information, which is often a physician's office. Staff in the physician offices will be provided with a list of charts to pull and will do this prior to the nurse reviewer's visit. Each chart will be individually reviewed page by page and the nurse reviewer will identify potential additional diagnosis codes not currently documented.

The nurse reviewers are trained to recognize many situations in which valid RAPS diagnosis codes have not been documented and submitted. Some of the reasons that valid RAPS codes may not have been used in the medical record being reviewed include:
1) A primary care physician will often have a practice of only using a limited set of diagnosis codes when additional diagnoses are appropriate.
2) If the patient was seen by a specialist, the specialist will often have a practice of only using a limited set of diagnosis codes when additional diagnoses are appropriate.
3) A radiologist does not enter a diagnosis code or submit a request for reimbursement, but instead often submits back to the ordering physician whatever initial code was on the request for radiological services at the time of ordering. The diagnosis code submitted on the request may be general and may not be accurate.
4) If a patient has received health services in a hospital, the hospital may have submitted a limited number of codes when additional diagnoses are appropriate.
5) Old diagnoses are not converted to new diagnoses where appropriate. For example, a patient with a the past diagnosis of acute myocardial infarction (“MI”) can appropriately have a diagnosis in subsequent years of old MI or angina, both of which are RAPS diagnosis codes.
6) Patient is not seen for an active problem in the current year, but the RAPS diagnosis could still be valid.
7) Diagnosis codes may be excluded or cut off due to space availability limitations in the provider's medical record systems.
8) Providers may not list all appropriate diagnosis codes addressed at a given visit.
9) Providers may not recognize that valid RAPS diagnosis codes exist for conditions that are stable or currently on treatment.
10) Providers often do not recognize that valid RAPS diagnosis codes exist for conditions that are not symptomatic.
11) If a patient has an inpatient stay at a hospital, inpatient problems are not addressed on an outpatient basis and are therefore not coded.
12) Underlying diagnoses are not recognized. For example, atrial fibrillation is a valid RAPS diagnosis code even after the insertion of a pacemaker.
13) Conditions are not accurately coded. For example, general diagnosis of atherosclerotic cardiovascular disease (not a RAPS diagnosis code) may be used when the patient has a valid RAPS diagnosis of old MI or angina.
14) If a specialist has made a RAPS diagnosis, but the diagnosis is not within his or her specialty and the specialist does not code that diagnosis even though he made that diagnosis.
15) A valid RAPS diagnosis code may not have been used because the person entering the diagnosis on the medical record chose other diagnosis codes deemed more serious.
16) A physician's staff does not accurately enter all codes diagnosed by the provider.
17) Data entry personnel do not enter all codes accurately.

The preferred embodiment of the present method and system classifies some diagnosis codes as permanent, lapsing, variable, or changing. These classifications assist the nurse reviewer in recognizing where a RAPS diagnosis code is appropriate. A permanent code would be applicable as long as the patient lives. A lapsing code would have a finite life span, for example, an acute MI of 8 weeks, or a cancer as long as it was under active treatment. A variable code could be either permanent or lapsing, depending on the situation. A changing code is one that changes into a different code over time, for example, a diagnosis of acute MI can in subsequent years appropriately change to old MI or angina. It may be important for the reviewer to analyze the type of code to assure correct coding on an ongoing basis both for adding, changing and deleting codes. In a preferred embodiment, many of these classified codes are maintained in a format to which the nurse reviewer can refer during the review and which suggests to the nurse reviewer appropriate RAPS codes based on the code entered in the patient health related data under review.

Thus, upon completion of the review of a patient's health related information, there can be up to three outcomes 4. First, the patient's health related information can be complete, and include all health status data necessary under the RAPS methodology. In such case, the health status data is captured 5 and submitted 11 for further use or processing.

Second, the health related information can have appropriate diagnoses identified, but lack evidence that all such diagnoses have been addressed. Third, the health related information can lack some appropriate diagnoses. In either the second or third outcome, possible additional appropriate health status data is suggested 6 to the physician. In a preferred embodiment, that data can be a diagnosis, or a suggestion to the physician that certain diagnoses be addressed with the patient, or both.

In a preferred embodiment, the nurse reviewer will identify all potential RAPS diagnosis codes and will note the location and date in the patient's health related information of the piece of information that may justify the use of the RAPS diagnosis, for example, cardiology consult on Jun. 10, 2003. The nurse reviewer then generates a form document upon completion of the review of a patient's health related data. This form, intended for use by the physician the next time the patient visits the physician, informs the physician of possible additional health status data.

Once presented with the suggested additional data, the physician makes the decision 7 to agree or disagree with, or accept or reject, the additional data. The above described form may provide the physician with the opportunity indicate such agreement/acceptance or disagreement/rejection. The form may also state that the physician has examined the patient, formulated a treatment plan, and discussed his or her findings and recommendations with the patient concerning any diagnoses listed on the sheet with which the physician agrees.

If the physician disagrees with or rejects the suggested additional data, the decision is captured 8 in the patient's health related information. That decision is not processed further 9, except that in a preferred embodiment, it may be the subject of a report generated by a software application. If the physician agrees with or accepts the suggested additional health status data, then the additional data is captured 10 and submitted 11 for further use or processing.

In a preferred embodiment, an ongoing notification process back to the physicians may be used to remind them of the forms not yet returned in a timely period and patients not yet seen. These reminders will also be accompanied by follow up calls when necessary and physicians' review and compliance will be monitored and reported. Upon submission of the completed forms the physician will be reimbursed an amount based on the number of forms the physician completes and returns. Alternative methods of reimbursement for physicians may be considered in the future including a quality bonus for meeting overall audit scores and accuracy of diagnoses.

As disclosed above, the submission 11 of the data contemplates further use or processing. One such use of the health status data is submission of the data to a payor for payment. Other uses could include continued tracking of patients' health status, generation of reports, quality improvement (quality of care), quality control (quality of compliance), analysis of treatment protocols, disease tracking, and wellness management.

The process will be repeated on an annual basis 12 with analysis conducted by the nurse reviewers using the same process as previously described.

In a preferred embodiment of the present invention, the method or system is aided by a database application. The application may have the following characteristics:
1) Data matching and validation engine;
2) Database of HCC ICD9 codes and all other ICD9 codes with simplified terminology;
3) Nurse Reviewer data input form;
4) Clerical data input form from RAPS data attestation sheets; and
5) Reporting module, which includes:
   a) Nurse reviewer work sheets and forms;
   b) Physician reminder and follow up forms;
   c) Provider reports;
   d) Health plan reports;
   e) Economic reports; and
   f) Management reports.

It is useful to examine how an embodiment of the present invention is used in practice in connection with a collection of patient health related information. Initially, patient health related information is gathered from all of the provider's or health plan's data systems. These are compiled and, from that the provider's or the health plan's main data set is established. The provider or health plan then pulls a complete list of all the patients for a particular facility clinic and puts it on a shared administration drive. Also, using a standard scheduling application, the provider or health plan identifies patients who are scheduled for appointments for the following two weeks and places that list on the shared administration drive. The facilities then pull a minimum of 50 records a day for the nurse reviewers to review. The nurse reviewers then go to the facilities to review the records as described above.

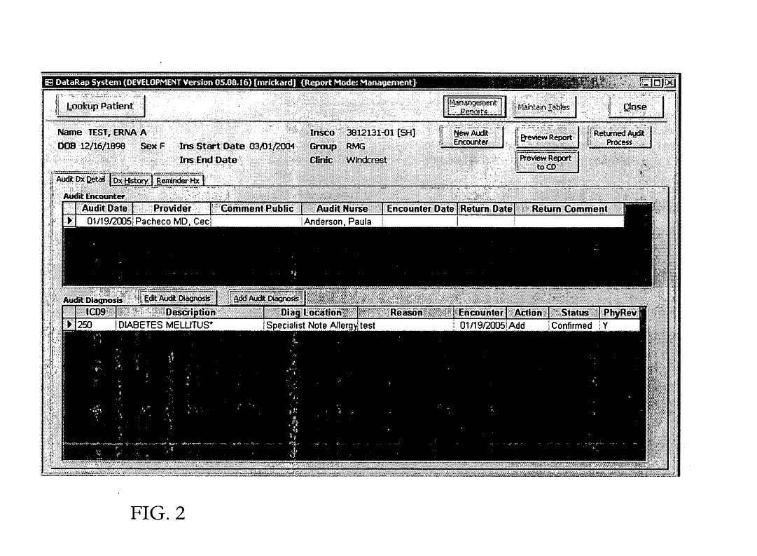

Once a database application is obtains the patient's health related information or gains access to the information on a shared storage medium, each patient is allocated a record that shows the patient's demographic information, diagnosis detail, diagnosis history, and history of any physician reminders. FIG. 2 is an exemplary screen view of an application showing a “Lookup Patient” screen, from which the user can obtain various patient information, demographics, audit diagnosis history, diagnosis history, and physician/nurse reminder history.

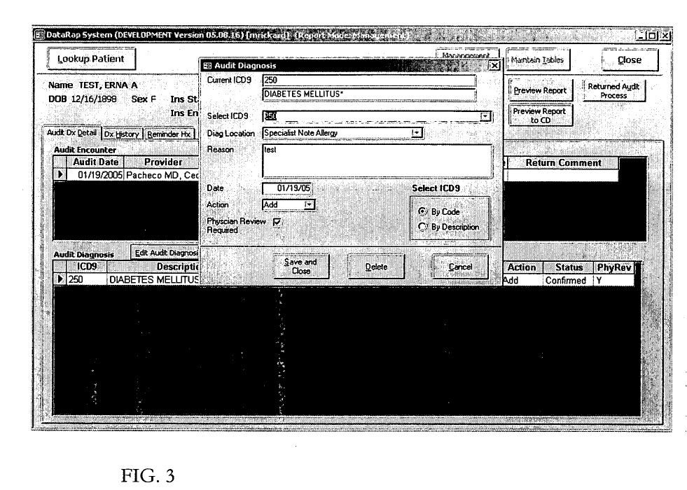

In the course of the review of the patient's health related data, the nurse reviewer may determine that an appropriate diagnosis is absent. From the “Lookup Patient” screen, the reviewer can audit diagnoses and suggest diagnoses. FIG. 3 shows an exemplary screen view of an application showing an “Audit Diagnosis” dialog box, allowing the review to select an ICD9 code, explain the reason for the selection of the code, and indicate whether physician review is required.

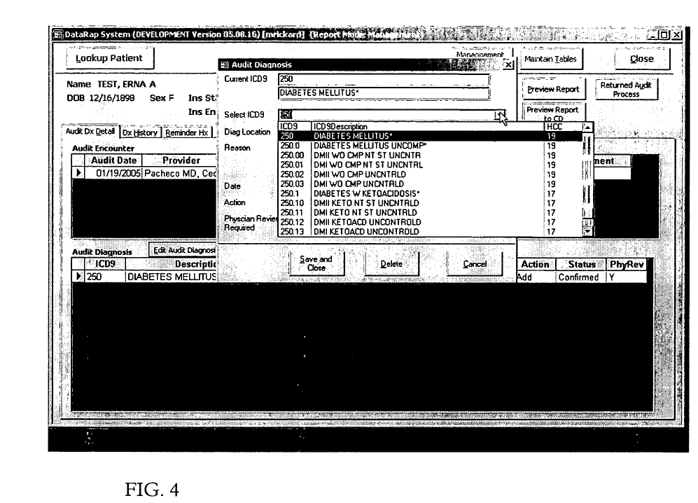

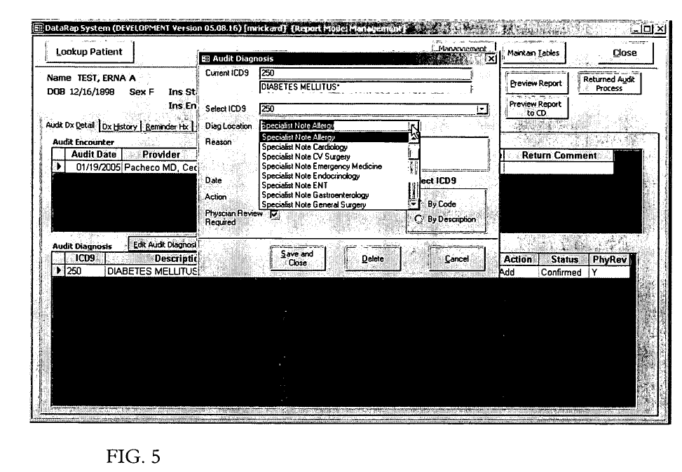

An application may assist the reviewer in determining the appropriate diagnosis by providing a drop-down menu of possible appropriate diagnoses in the “Audit Diagnosis” dialog box as shown in FIG. 4. The “Audit Diagnosis” dialog box may also provide a drop-down menu to assist the user in identifying the location in the patient's health related data of the evidence supporting the suggested diagnosis. FIG. 5.

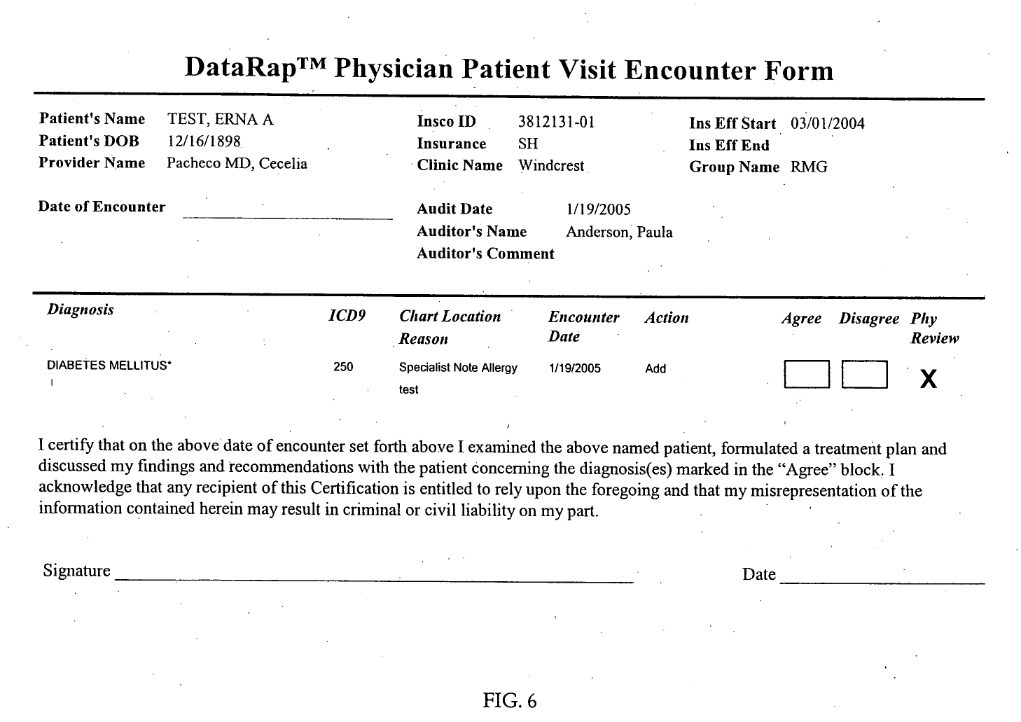

If after the review of the patient's health related data it is determined that appropriate health status data is lacking, the reviewer can use an application to generate a “Physician Patient Visit Encounter Form” FIG. 6 which is placed in the provider's medical records for the physician to examine and address with the patient, if appropriate, on the patient's next visit. An application can also generate a “Nurse Patient Visit Encounter Form” that is used in a similar manner and for a similar purpose.

The form FIG. 6 may include patient identifying information and list additional suggested diagnoses. The form may also provide the physician with check boxes to indicate agreement or disagreement with the additional diagnoses as well as additional information, such as an attestation that the physician has examined the patient, formulated a treatment plan, and discussed the physician's findings and recommendations with the patient concerning all diagnoses for which the physician marked the “Agree” check box. If the physician agrees with the suggested diagnoses, he or she will also address these diagnoses in the medical record progress note on the day of that visit.

After the physician completes the form, the form will be returned for further processing. The forms may be barcode labeled for ease of data entry. The patient records may then be retrieved in a database application and the codes to which the physician attested as valid and accurate will be noted as such, those which are not valid and active according to the physician will also be so noted. The data on active RAPS codes will then be submitted to the health plan or to Medicare or other payor directly, for reimbursement.

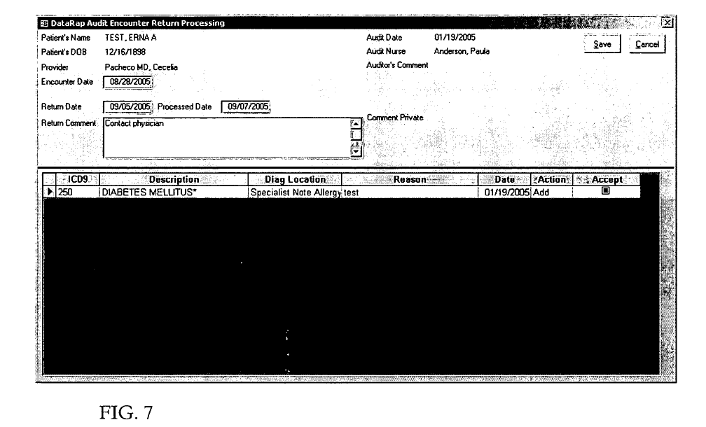

For each patient, an application may track the status of the Physician Patient Visit Encounter Form submitted to the physician. FIG. 7 is an exemplary “Audit Encounter Return Processing” screen showing whether the physician has accepted or rejected the proposed additional health status information identified in the form FIG. 6.

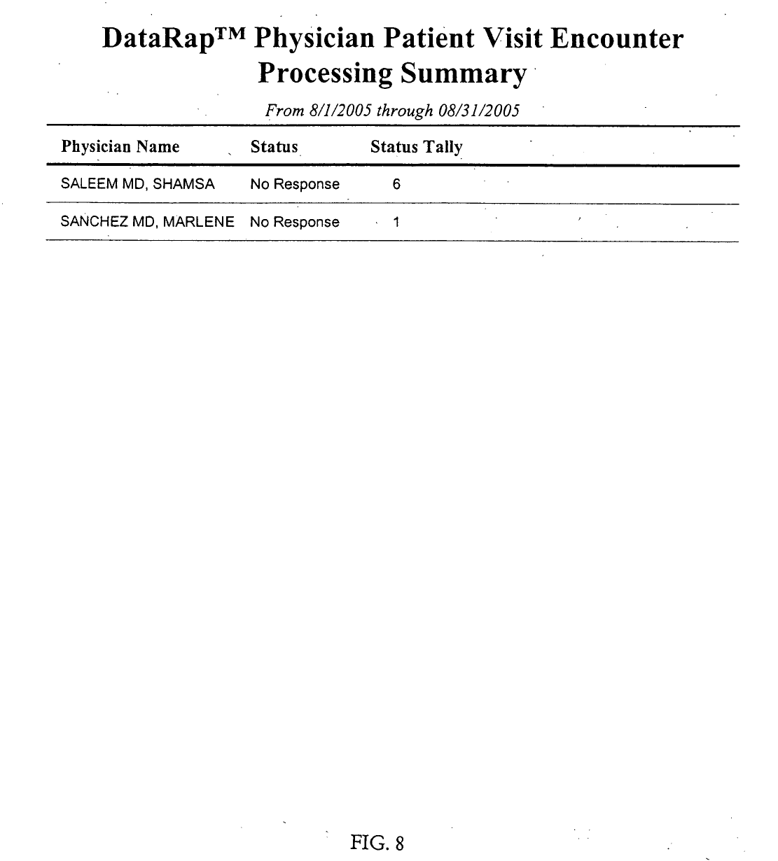

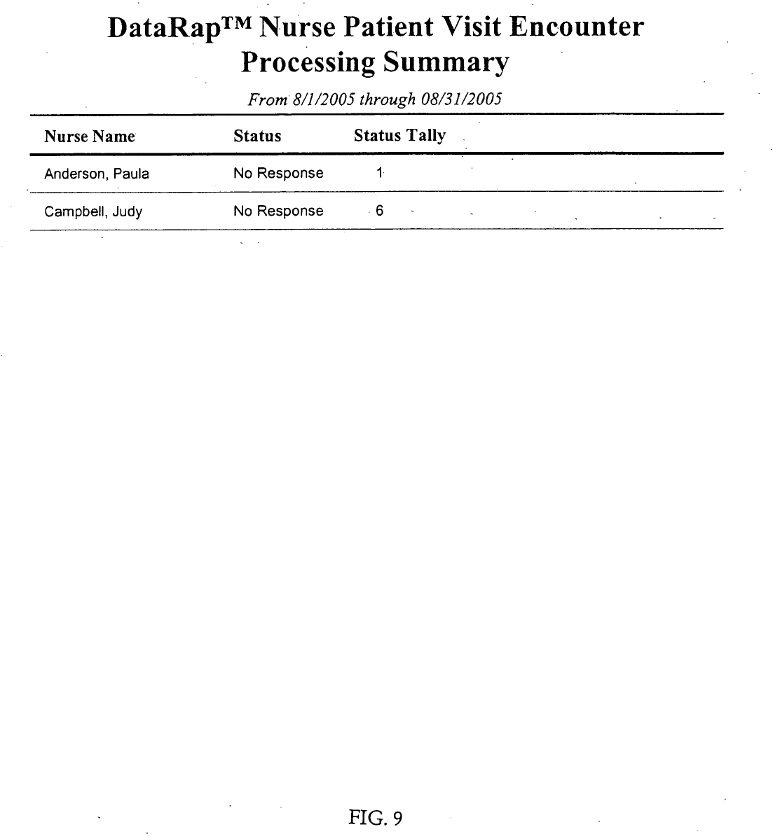

An application may also enable tracking of the responses to the Physician Patient Encounter Forms by physician or nurse, rather than by patient. FIG. 8 is a “Physician Patient Visit Encounter Processing Summary” generated by an application. It shows, for each physician, the status of the physician's responses to the Physician Patient Visit Encounter Forms submitted to the physician. FIG. 9 is an example of a similar report generated by an application describing nurse responses to the Nurse Patient Visit Encounter Forms.

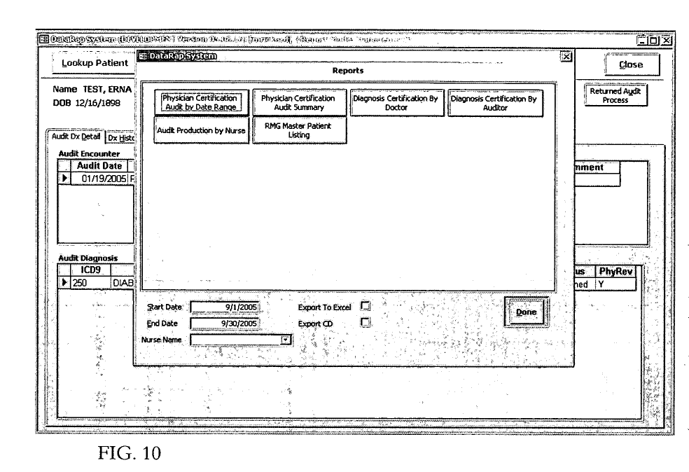

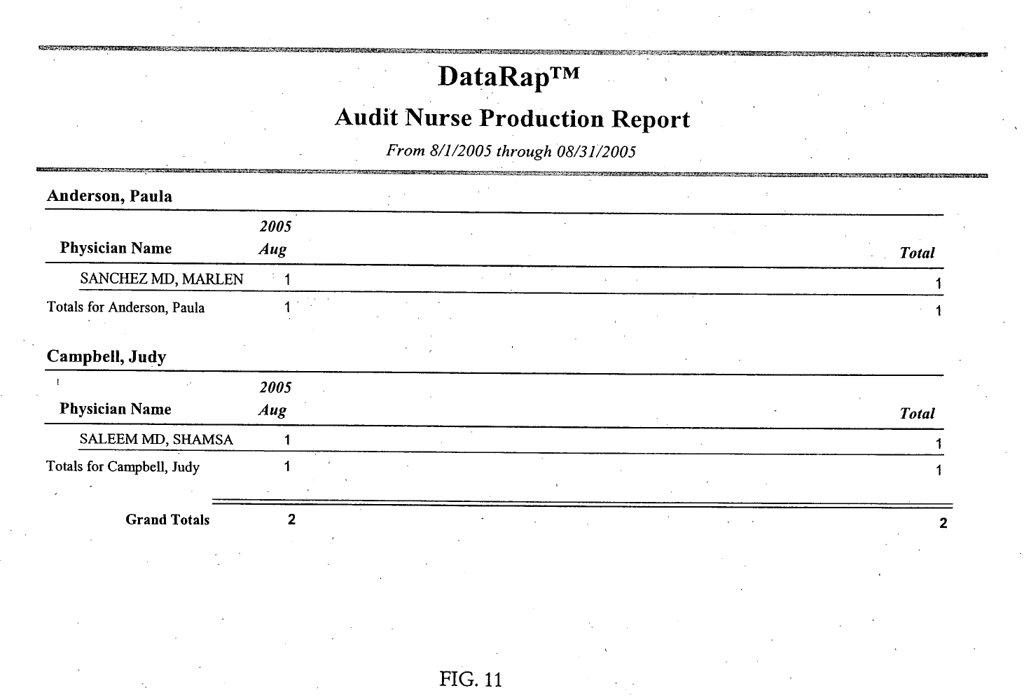

An application may have a reporting capability. FIG. 10 is an exemplary screen view showing a “Reports” dialog box, from which the user can generate various reports, such as Physician Certification Audit by Date Range, Physician Certification Audit Summary, Diagnosis Certification by Doctor, Diagnosis Certification by Auditor, master patient listings, and Audit Production by Nurse. FIG. 11 is an example of an Audit Nurse Production Report generated from the “Reports” dialog box.

In addition to the reviewing, tracking, and reporting capabilities, an application may be able to communicate directly with electronic medical records systems or other health care provider information system software to share information. An application may also be capable of electronically transmitting the health related data to the appropriate payor for processing.

Patient data may remain on an application, and be supplemented on at least an annual basis. This feature would enable the provider to develop a meaningful database and history of patient health status. An application may then be capable of further analysis of this patient information for various purposes, such as quality improvement, quality control, analysis of treatment protocols, disease tracking, and wellness management.

In one embodiment, the database application can retrieve patient health data directly from an electronic medical records system or health care provider information system software application. Upon completion of the review of each patient's medical record, the database application will communicate with the electronic medical records system or information system software application to include the physician approved additional health status information.

The preferred embodiment of the present invention generally relates to the managed care model as it currently exists. However, a person of skill in the art would readily appreciate that the present method and system would be useful in other health care environments that uses a RAPS-type risk assessment and adjustment system. Furthermore, the present method and system can also be applied to the fee for service model to the extent that payment in that model depends on maintaining and monitoring patients' health status data and using coding systems.

What is claimed is:

1. A method for managing patient health status data comprising:
collecting all available health related information for a patient from one or more sources;
analyzing said patient's health related information to determine whether a set of appropriate health status data is present in said patient's health related information;
identifying in said patient's health related information all of said set of appropriate health status data that is unreported in or inconsistent with said patient's health-related information;
suggesting to said patient's physician that said patient's health related information be supplemented as appropriate to correct any of said patient's health status data that is unreported in or inconsistent with said patient's health-related information, and obtaining physician approval or rejection of said suggested supplementation; and
compiling said patient's health status data for future use.

2. The method of claim 1 wherein said health related information comprises patient medical records, health plan records, pharmacy benefit managers records, physician or health care facility billing data, or laboratory results.

3. The method of claim 1 wherein said health related information is maintained by a health care provider or a health maintenance organization.

4. The method of claim 1 wherein said health related information is maintained by a health care provider or a health maintenance organization providing Medicare/Medicaid health care services.

5. The method of claim 1 wherein said analysis of all available health related information for a patient to determine whether all appropriate health status data is present in said patient's health related information is performed by a nurse reviewer with significant clinical experience and training in coding systems.

6. The method of claim 1 wherein said analysis of all available health related information for a patient to determine whether all appropriate health status data is present in said patient's health related data is performed at least in part by a software application.

7. The method of claim 1 wherein said identification in said patient's health related information all of said set of appropriate health status data that is unreported in or inconsistent with said patient's health-related information is performed by a nurse reviewer with significant clinical experience and training in coding systems.

8. The method of claim 1 wherein said identification in said patient's health related information all of said set of appropriate health status data that is unreported in or inconsistent with said patient's health-related information is performed at least in part by a software application.

9. The method of claim 1 wherein said health status data comprises diagnosis, treatment, and physician-patient encounter information.

10. The method of claim 1 wherein said health status data comprises the data required for provider payment under the Center For Medicare/Medicaid Services Risk Adjusted Payment System.

11. The method of claim 1 wherein said future use of said patient's health status data comprises submission of the data to a health care services payor, maintenance of the data, analysis of the data, and manipulation of the data to generate reports.

12. The method of claim 1 wherein said compiling said patient's health status data for future use is performed at least in part by a software application.

13. A system for managing patient health status information, comprising:
a means for collecting all available health related information for a patient from one or more sources;
a means for analyzing said patient's health related information to determine whether a set of appropriate health status data is present in said patient's health related data;
a means for identifying in said patient's health related information all appropriate health status data that is unreported in or inconsistent with said patient's health-related information;
a means for suggesting to said patient's physician that said patient's health related information be supplemented as appropriate to correct any of said patient's health status data that is unreported or inconsistent with said patient's health-related information, and obtaining physician approval or rejection of said suggested supplementation; and
a means for compiling said patient's health status data for future use.

14. The system of claim 13 wherein said health related information comprises patient medical records, health plan records, pharmacy benefit managers records, physician or health care facility billing data, or laboratory results.

15. The system of claim 13 wherein said health related information is maintained by a health care provider or a health maintenance organization.

16. The system of claim 13 wherein said health related information is maintained by a health care provider or a health maintenance organization providing Medicare/Medicaid health care services.

17. The system of claim 13 wherein said means of analyzing of all available health related information for a patient to determine whether all appropriate health status data is present in said patient's health related information is a nurse reviewer with significant clinical experience and training in coding systems.

18. The system of claim 13 wherein said means for analyzing all available health related information for a patient to determine whether all appropriate health status data is present in said patient's health related data is performed a software application.

19. The system of claim 13 wherein said means for identifying in said patient's health related information all of said set of appropriate health status data that is unreported in or inconsistent with said patient's health-related information is a nurse reviewer with significant clinical experience and training in coding systems.

20. The system of claim 13 wherein said means for identifying in said patient's health related information all of said set of appropriate health status data that is unreported in or inconsistent with said patient's health-related information is a software application.

21. The system of claim 13 wherein said health status data comprises diagnosis, treatment, and physician-patient encounter information.

22. The system of claim 13 wherein said health status data comprises the data required for provider payment under the Center For Medicare/Medicaid Services Risk Adjusted Payment System.

23. The system of claim 13 wherein said future use of said patient's health status data comprises submission of the data to a health care services payor, maintenance of the data, analysis of the data, and manipulation of the data.

24. The method of claim 13 wherein said means for compiling said patient's health status data for future use is a software application.

25. A software application comprising:
computer code for analyzing all available health related information for a patient and storing a set of appropriate health status data for said patient;
computer code adapted to access said set of appropriate health status data and identify a set of suggested modifications relative to inaccuracies or incompleteness in said set of appropriate health status data;
computer code for receiving said set of suggested modifications and adapted to generate a request for physician approval or rejection of said set of suggested modifications;
computer code adapted to store said physician approved modification to said patient's health status data; and
computer code adapted to access said patient's health status data and adapted to perform additional processes on said patient's health status data.

26. The software application of claim 25 wherein said health related information comprises patient medical records, health plan records, pharmacy benefit managers records, physician or health care facility billing data, or laboratory results.

27. The software application of claim 25 wherein said health related information is maintained by a health care provider or a health maintenance organization.

28. The software application of claim 25 wherein said health related information is maintained by a health care provider or a health maintenance organization.

29. The software application of claim 25 wherein said health related information is maintained by a health care provider or a health maintenance organization providing Medicare/Medicaid health care services.

30. The software application of claim 25 wherein said health status data comprises diagnosis, treatment, and physician-patient encounter information.

31. The software application of claim 25 wherein said health status data comprises the data required for provider payment under the Center For Medicare/Medicaid Services Risk Adjusted Payment System.

32. The software application of claim 25 wherein said additional processes performed on said patient's health status data comprises submission of the data to a health care services payor, maintenance of the data, analysis of the data, and manipulation of the data to generate reports.
